(19) United States
(12) Patent Application Publication
(10) Pub. No.: US 2018/0263520 A1
(43) Pub. Date: Sep. 20, 2018

(54) Integrated Physiological Signal Acquisition System

(71) Applicant: VitalSigns Technology Co., Ltd., Hsinchu County (TW)

(72) Inventor: Jih-Liang JUANG, Hsinchu County (TW)

(21) Appl. No.: 15/654,730

(22) Filed: Jul. 20, 2017

(30) Foreign Application Priority Data
Mar. 17, 2017 (CN) 201710160978.8

(51) Int. Cl. A61B 5/0402; G06F 19/00; A61B 5/00; A61B 5/04

(52) U.S. Cl. CPC A61B 5/0402; A61B 5/04004; A61B 5/72; G06F 19/3406

(57) ABSTRACT

An integrated physiological signal acquisition system comprises an analysis host, a physiological signal acquisition element and at least one biological sensor coupled to the physiological signal acquisition element for sensing a physiological signal of a user. The physiological signal is transmitted to the physiological signal acquisition element. The physiological signal acquisition element transmits the physiological signal to the analysis host for analysis. The at least one biological sensor is a pluggable biological sensor. Accordingly, only an analysis host and a physiological signal acquisition element are need to you realize different physiological signal detection, thus, the cost of hardware can be significantly reduced.

### RELATED APPLICATIONS

This application claims priority to China Application Serial Number 201710160978.8, filed Mar. 17, 2017, which is herein incorporated by reference.

### BACKGROUND

### Field of Invention

The present invention relates to a physiological signal acquisition system. More particularly, the present invention relates to an integrated physiological signal acquisition system.

### Description of Related Art

Today, the rapid development of the economy causes an equally rapid pace of life. Therefore, people often ignore the importance of health. A poor diet and lack of exercise are the especially major health worries for the modern people.

Accordingly, many different types of physiological signal acquisition systems have been developed to help users to detect their own physiological conditions to understand health status at home. However, traditional physiological signal acquisition system always includes a fixed type of biological sensors, such as an ECG sensor, an EMG sensor or a heartbeat sensor, to detect those physiological signals. Then, the detected result is displayed on the screen disposed on the physiological signal acquisition system or printed with the built-in thermal paper. Therefore, it is necessary to use different physiological signal acquisition systems to detect different physiological signals. Such physiological signal acquisition systems are not only expensive but also difficult to integrate the detected data. Therefore, how to reduce the detection cost and provide integrated data become the pursuit goal.

### SUMMARY

The present disclosure provides an integrated physiological signal acquisition system. The system comprises an analysis host, a physiological signal acquisition element and at least one biological sensor coupled to the physiological signal acquisition element for sensing a physiological signal of a user. The physiological signal is transmitted to the physiological signal acquisition element. The physiological signal acquisition element transmits the physiological signal to the analysis host for analysis. The at least one biological sensor is a pluggable biological sensor.

In an embodiment, the physiological signal acquisition element further comprising a communication module, a physiological signal processing module, and at least one connection interface for connecting the at least one biological sensor, wherein the at least one biological sensor transmits the physiological signal to the physiological signal processing module through the at least one connection interface, and transmits to the analysis host through the communication module.

In an embodiment, the physiological signal is an analog type of physiological signal, and the physiological signal processing module converts the analog type of physiological signal into a digital type of physiological signal.

In an embodiment, the at least one biological sensor comprises a plurality of biological sensors, and the at least one connection interface comprises a plurality of connection interfaces for connecting the plurality of biological sensors respectively.

In an embodiment, the analysis host has a plurality of analysis software for analyzing the physiological signals detected by the biological sensors respectively.

In an embodiment, the communication module connects the analysis host by wired.

In an embodiment, the communication module connects the analysis host by wired through USB interface, Lightening interface, SATA interface, PCI-E interface, UART interface, SPI interface, I2C interface, I2S interface, or PCM interface.

In an embodiment, the at least one biological sensor is selected from the group consisting of a sensor for detecting blood glucose (GLUCOSE) signal, a sensor for detecting electrocardiogram (ECG) signal, a sensor for detecting Photoplethysmography (PPG) signal, a sensor for detecting thermal signal, a sensor for detecting an electromyography (EMG) signal, a sensor for detecting respiration signal, a sensor for detecting brainwave (EEG) signal, a sensor for detecting pressure signal and a combination thereof.

In an embodiment, the analysis host starts a corresponding software to analyze the physiological signal based on a packet data transmitted by the at least one biological sensor.

In an embodiment, the system further comprises a hand-held device for receiving an analysis result from the analysis host.

In conclusion, the present invention uses a single biological sensor or simultaneously use multiple biological sensors to couple the same physiological signal acquisition element for physiological signal detection. The detected physiological signal is transmitted to the remote analysis host for analyzing. Therefore, only a single analysis host and a single physiological signal acquisition element are needed. The hardware cost can be significantly reduced.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiments, with reference made to the accompanying drawings as follows:

FIG. 1 illustrates a schematic diagram of an integrated physiological signal acquisition system according to an embodiment of the present invention;

FIG. 2 is a schematic diagram of a physiological signal acquisition element according to an embodiment of the present invention; and

FIG. 3 is a schematic diagram of a physiological signal acquisition element according to another embodiment of the present invention.

### DETAILED DESCRIPTION

In order to make the description of the disclosure more detailed and comprehensive, reference will now be made in detail to the accompanying drawings and the following embodiments. However, the provided embodiments are not used to limit the ranges covered by the present disclosure. Moreover, the order of any steps described is not used to limit the execution sequence thereof. Any device capable of achieving an equivalent effect through rearrangement is also covered by the present disclosure.

The terminology used herein is for the purpose of describing particular embodiments only and is not intended to be limiting of the invention. As used herein, the singular forms “a,” “an” and “the” are intended to include the plural forms as well, unless the context clearly indicates otherwise.

In this document, the term “coupled” may also be termed as “electrically coupled,” and the term “connected” may be termed as “electrically connected.” “Coupled” and “connected” may also be used to indicate that two or more elements cooperate or interact with each other.

The present invention provides an integrated physiological signal acquisition system. Different types of biological sensor may electrically connect to a same physiological signal acquisition element. By using this physiological signal acquisition element, the biological information captured by these biological sensors is transmitted to a same analysis host with different analysis software for analyzing. Therefore, it is not necessary to use different analysis hosts to present different biological information. Such integrated physiological signal acquisition system not only has a lower system cost but also can integrate different biological information for further analyzing.

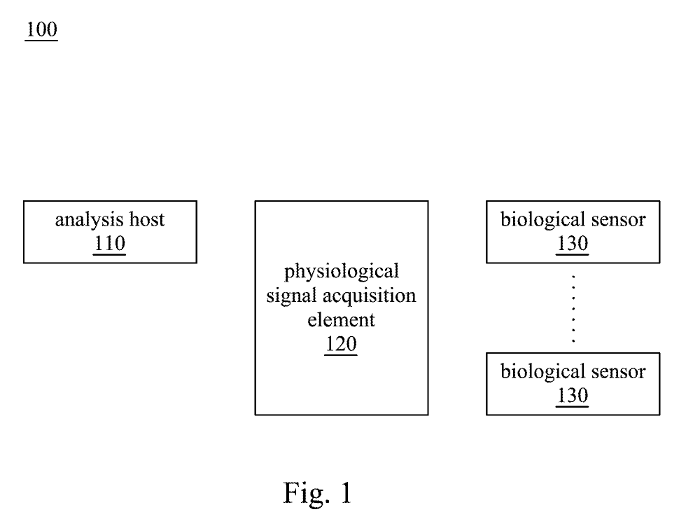

FIG. 1 illustrates a schematic diagram of an integrated physiological signal acquisition system according to an embodiment of the present invention. The integrated physiological signal acquisition system 100 comprises an analysis host 110, a physiological signal acquisition element 120 and many biological sensors 130. The analysis host 110 communicates with the physiological signal acquisition element 120 by wired or wireless. The biological sensors 130 are selected from the group consisting of sensors for detecting blood glucose (GLUCOSE) signal, sensors for detecting electrocardiogram (ECG) signal, sensors for detecting Photoplethysmography (PPG) signal, sensors for detecting thermal signal, sensors for detecting an electromyography (EMG) signal, sensors for detecting respiration signal, sensors for detecting brainwave (EEG) signal, sensors for detecting pressure signal and a combination thereof. However, other type of sensors is also used in this embodiment. These biological sensors 130 are coupled to the physiological signal acquisition element 120 for sensing physiological signals of a user. The physiological signals are transmitted to the analysis host 110 through the physiological signal acquisition element 120 by wired or wireless. The wired transmission method may use an USB interface, a Lightening interface, a SATA interface, a PCI-E interface, an UART interface, a SPI interface, an I2C interface, an I2S interface or a PCM interface for data transmission. The wireless transmission method may use a blue-tooth interface, or wireless internet interface for data transmission. The analysis host 110 has different physiological signal analysis software to perform immediate analysis of the physiological signals detected by the corresponding biological sensors 130. 130. For example, when the biological sensor 130 connected to the physiological signal acquisition element 120 is a sensor for detecting blood glucose, the detected blood glucose signal is transmitted to the analysis host 110 through the physiological signal acquisition element 120 by a wired or wireless manner. Then, the analysis host 110 starts the corresponding analysis software to analyze the blood glucose signal. In one embodiment, the analysis host 110 is a computer, a tablet or a remote server. If the analysis host 110 is a personal computer or a tablet, the result of the analysis of the physiological signal is immediately presented on the corresponding computer screen for the user's reference. On the other hand, if the analysis host 110 is a remote server, the result of the analysis of the physiological signal is immediately transmitted to the user's hand-held device for the user's reference.

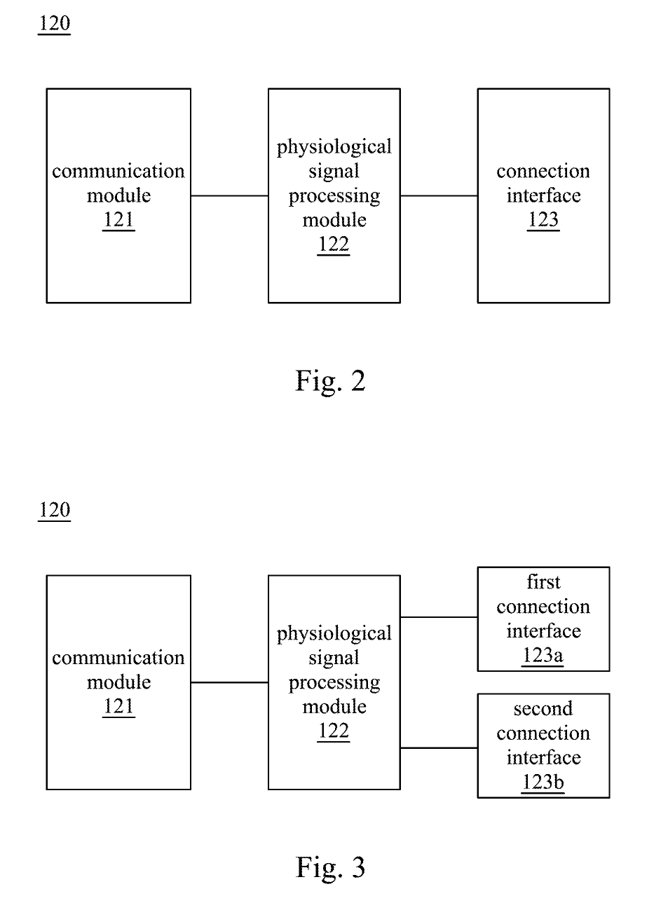

FIG. 2 is a schematic diagram of a physiological signal acquisition element according to an embodiment of the present invention. The physiological signal acquisition element 120 includes a communication module 121, a physiological signal processing module 122, and at least one connection interface 123. The connection interface 123 is used to connect with the biological sensor 130. The physiological signal detected by the biological sensor 130 is transmitted to the physiological signal processing module 122 through the connection interface 123. In an embodiment, if the biological sensor 130 is an analog type of biological sensor for generating an analog type of physiological signal, the physiological signal processing module 122 may convert the analog type of physiological signal into a digital type of physiological signal. Then, the physiological signal processing module 122 transmits the digital type of physiological signal to the remote analysis host 110 through the communication module 121 to process for analyzing the physiological condition of the user.

In another embodiment, if the biological sensor 130 is a digital type of biological sensor for generating a digital type of physiological signal, the biological sensor 130 may generate the physiological signal with binary value. Then, the processing module 122 may transmit the physiological signal with binary value to the remote analysis host 110 through the communication module 121 to process for analyzing the physiological condition of the user. The biological sensor 130 is a pluggable biological sensor. That is, the user can detect different physiological signals by exchanging the biological sensors 130. For example, the user may connect the biological sensor 130 for detecting blood glucose (GLUCOSE) signal to the connection interface 123 to monitor a user's blood glucose, then, transmit the detected result through the communication module 121 to the analysis host 110. After that the biological sensor 130 for detecting blood glucose (GLUCOSE) signal is exchanged to another biological sensor 130 for detecting Photoplethysmography (PPG) signal to monitor the user's blood pressure. Then, the detected result is transmitted to the analysis host 110 through the communication module 121 as well. Accordingly, it is only necessary to use a single physiological signal acquisition element 120 and the analysis host 110 to simultaneously detect different physiological signals, thereby greatly reducing the hardware cost of detecting.

In another embodiment, the connection interface 123 may be plural, as shown in FIG. 3, including two connection interfaces, a first connection interface 123a and a second connection interface 123b. However, it is noticed that the FIG. 3 is only an example to explain the claimed invention. FIG. 3 is not intended to limit the claimed invention. In other embodiments, the number of connection interfaces may be modulated depending on the invention being applied. In this embodiment, the user may connect different biological sensors 130 to the connection interfaces 123a and 123b for simultaneously monitoring two physiological signals. For example, the first connection interface 123a may be connected to a biological sensor for detecting Photoplethysmography (PPG) signal, and the second connection interface 123b may be connected to a biological sensor for detecting breathing signal. Then, the two detected results are transmitted to the analysis host 110 through the communication module 121, respectively. In this way, a complete detection result can be obtained. That is, by simultaneously monitoring and analyzing a variety of physiological signals, users may use cross validation method to validate the results of different physiological signals to provide better detection and analysis.

The remote analysis host 110 may obtain the information of the current biological sensor 130 through the packet data. Accordingly, the analysis host 110 can start a corresponding analysis software to analyze the physiological signal according to the packet data. The information of the packet data includes, but not limited to, the number of biological sensors, the sampling frequency of the biological sensor, the order of data of the biological sensor, the number of data in the packet, the number of bits of the biological sensor data, and compression method of the biological sensor data.

The present invention can use a single biological sensor or simultaneously use multiple biological sensors to couple the same physiological signal acquisition element for physiological signal detection. The detected physiological signal is transmitted to the remote analysis host for analyzing. In other words, only a single analysis host and a single physiological signal acquisition element are needed. The hardware cost can be significantly reduced. Moreover, by coupling different biological sensors, different physiological signals may be captured.

Even though the present disclosure is disclosed as above, the disclosure is not used to limit the present disclosure. It will be apparent to those skilled in the art that various modifications and variations can be made to the present disclosure without departing from the spirit or scope of the invention; thus, it is intended that the range protected by the present disclosure should refer to the scope of the following claims.

What is claimed is:

1. An integrated physiological signal acquisition system, comprising:
an analysis host;
a physiological signal acquisition element; and
at least one biological sensor coupled to the physiological signal acquisition element for sensing a physiological signal of a user and transmitting the physiological signal to the physiological signal acquisition element,
wherein the physiological signal acquisition element transmits the physiological signal to the analysis host for analyzing,
wherein the at least one biological sensor is a pluggable biological sensor.

2. The integrated physiological signal acquisition system of claim 1, wherein the physiological signal acquisition element further comprising:
a communication module;
a physiological signal processing module; and
at least one connection interface for connecting the at least one biological sensor, wherein the at least one biological sensor transmits the physiological signal to the physiological signal processing module through the at least one connection interface, and transmits to the analysis host through the communication module.

3. The integrated physiological signal acquisition system of claim 2, wherein the physiological signal is an analog type of physiological signal, and the physiological signal processing module converts the analog type of physiological signal into a digital type of physiological signal.

4. The integrated physiological signal acquisition system of claim 2, wherein the at least one biological sensor comprises a plurality of biological sensors, and the at least one connection interface comprises a plurality of connection interfaces for connecting the plurality of biological sensors respectively.

5. The integrated physiological signal acquisition system of claim 1, wherein the analysis host has a plurality of analysis software for analyzing the physiological signals detected by the biological sensors respectively.

6. The integrated physiological signal acquisition system of claim 2, wherein the communication module connects the analysis host by wired.

7. The integrated physiological signal acquisition system of claim 6, wherein the communication module connects the analysis host by wired through USB interface, Lightening interface, SATA interface, PCI-E interface, UART interface, SPI interface, I2C interface, I2S interface, or PCM interface.

8. The integrated physiological signal acquisition system of claim 1, wherein the at least one biological sensor is selected from the group consisting of sensor for detecting blood glucose (GLUCOSE) signal, sensor for detecting electrocardiogram (ECG) signal, sensor for detecting Photoplethysmography (PPG) signal, sensor for detecting thermal signal, sensor for detecting an electromyography (EMG) signal, sensor for detecting respiration signal, sensor for detecting brainwave (EEG) signal, sensor for detecting pressure signal and a combination thereof.

9. The integrated physiological signal acquisition system of claim 1, wherein the analysis host starts a corresponding software to analyze the physiological signal based on a packet data transmitted by the at least one biological sensor.

10. The integrated physiological signal acquisition system of claim 1, wherein the system further comprises a hand-held device for receiving an analysis result from the analysis host.
